(19) United States
(12) Patent Application Publication
(10) Pub. No.: US 2017/0360791 A1
(43) Pub. Date: Dec. 21, 2017

(54) PHARMACEUTICAL COMPOSITIONS FOR INCREASING THE BIOAVAILABILITY OF POORLY SOLUBLE DRUGS

(71) Applicant: ASTEX PHARMACEUTICALS, INC., Dublin, CA (US)

(72) Inventors: Rajashree Joshi-Hangal, Dublin, CA (US); Sanjeev Redkar, Dublin, CA (US); Chunlin Tang, Dublin, CA (US)

(21) Appl. No.: 15/533,283

(22) PCT Filed: Dec. 4, 2015
(86) PCT No.: PCT/US15/63982

§ 371 Date: Jun. 5, 2017

Related U.S. Application Data
(60) Provisional application No. 62/087,643, filed on Dec. 4, 2014.

(51) Int. Cl. A61K 31/519; A61K 47/22; A61K 47/14; A61K 47/32; A61K 45/06

(52) U.S. Cl. CPC A61K 31/519; A61K 47/32; A61K 45/06; A61K 47/14; A61K 47/22

(57) ABSTRACT

Pharmaceutical compositions comprising poorly soluble compounds, such as BCS class II or class IV drugs (e.g. amuvatinib), are provided. The pharmaceutical compositions are effective for increasing the bioavailability of the compounds. Related kits and methods are also provided.

### CROSS-REFERENCE TO RELATED APPLICATIONS

The present application claims priority from the U.S. Provisional Patent Application No. 62/087,643 filed on Dec. 4, 2014 and titled “Pharmaceutical Compositions for Increasing the Bioavailability of Poorly Soluble Drugs”, the disclosure of which is incorporated herein by reference.

### BACKGROUND OF THE INVENTION

### Field of the Invention

The present invention is generally directed to novel pharmaceutical compositions for increasing the bioavailability of poorly soluble drugs, such as amuvatinib and salts thereof, and methods for their preparation and use as therapeutic or prophylactic agents.

### Description of the Related Art

Many pharmaceutically active compounds have limited solubility in water. This hydrophobic property often makes it difficult to formulate a drug so that it exhibits a satisfactory bioavailability profile in vivo. Poor bioavailability may lead to ineffective therapy, the need for higher dosing, erratic pharmacokinetics, and/or undesirable side effects.

It is difficult to provide an oral dosage form for drugs of limited solubility. A drug of poor water-solubility will not dissolve in water or gastrointestinal milieu readily which tends to lead to low availability of the drug to the surface of the absorbing tissue and low drug blood concentration. Compared to more soluble drugs, it is often difficult to sequester a sufficient amount of the poorly soluble drug in the dosage form (such as a tablet), especially when the drug requires a high dose.

The Biopharmaceutics Classification System (BCS) is a system developed to differentiate drugs based on their solubility and permeability, and can be used for predicting intestinal absorption of the drug. The BCS system categorizes drugs into four classes based on their solubility and intestinal permeability: class I drugs have high permeability and high solubility; class II drugs have high permeability and low solubility; class III drugs have low permeability and high solubility; and class IV drugs have low permeability and low solubility. Accordingly, formulation of BCS class II and IV drugs (i.e., poorly soluble drugs) presents significant challenges in the pharmaceutical industry.

Amuvatinib (and its pharmaceutically acceptable salts, e.g., HCl) is an orally bioavailable multi-targeted tyrosine kinase inhibitor, which is a potent inhibitor of mutant c-Kit and PDGFRα. Amuvatinib is also active as an inhibitor of DNA repair protein Rad51 following chemotherapy. Amuvatinib has shown synergistic activity with DNA damaging chemotherapy in several xenograft models and in a phase Ib combination study. Amuvatinib is also a poorly soluble BCS class IV drug, and improved oral formulations of Amuvatinib with increased bioavailability are thus desirable.

Accordingly, while progress has been made in this field, there remains a need in the art for improved formulations for poorly soluble drugs, such as amuvatinib. The present invention fulfills this need and provides further related advantages.

### BRIEF SUMMARY OF THE INVENTION

In brief, the present invention is generally directed to improved formulations of poorly soluble drugs, such as BCS class II and class IV drugs. In more particular embodiments, improved formulations of amuvatinib, and its pharmaceutically acceptable salts are provided, such as the HCl salt. The present inventors have unexpectedly found that the solubility, and thus bioavailability, of BCS class II and class IV drugs, such as amuvatinib, is synergistically increased by the combination of a lipid vehicle and a surfactant polymer. Accordingly, the presently disclosed formulations are effective to significantly decrease the daily pill burden (i.e., number of pills required per day) for patients on oral dosing regimens of BCS class II or class IV drugs and other poorly soluble drugs.

In one embodiment, a pharmaceutical composition is provided, the pharmaceutical composition comprising:

a) amuvatinib, or a stereoisomer, tautomer, pharmaceutically acceptable salt or prodrug thereof;

b) a surfactant polymer, or a pharmaceutically acceptable salt thereof;

c) a tocopherol, or a pharmaceutically acceptable salt thereof; and

d) a fatty acid, a fatty acid ester, or a pharmaceutically acceptable salt thereof.

In another embodiment, the present disclosure provides a kit comprising a solid dispersion and a vehicle in separate packages, wherein:
a) the solid dispersion comprises amuvatinib, or a stereoisomer, tautomer, pharmaceutically acceptable salt or prodrug thereof, and a surfactant polymer, or pharmaceutically acceptable salt thereof; and
b) the vehicle comprises a tocopherol, or pharmaceutically acceptable salt thereof, and a fatty acid, a fatty acid ester, or pharmaceutically acceptable salt thereof.

Other embodiments are directed to a kit comprising an active drug and a vehicle in separate packages, wherein:

a) the active drug is amuvatinib, or a stereoisomer, tautomer, pharmaceutically acceptable salt or prodrug thereof; and

b) the vehicle comprises a surfactant polymer, or pharmaceutically acceptable salt thereof, a tocopherol, or pharmaceutically acceptable salt thereof, and a fatty acid or fatty acid ester, or pharmaceutically acceptable salt thereof.

In another embodiment, a method for increasing the bioavailability of amuvatinib is provided, the method comprising preparing a pharmaceutical composition comprising:

a) amuvatinib, or a stereoisomer, tautomer, pharmaceutically acceptable salt or prodrug thereof;

b) a surfactant polymer, or a pharmaceutically acceptable salt thereof;

c) a tocopherol, or a pharmaceutically acceptable salt thereof; and

d) a fatty acid or fatty acid ester, or a pharmaceutically acceptable salt thereof.

Methods for treatment of cancer, comprising administering to a subject in need thereof a therapeutically effective amount of any of the disclosed pharmaceutical compositions are also provided. Additional disclosed methods include, a method for the treatment of a protein kinase-mediated disease, the method comprising administering to a subject in need thereof a therapeutically effective amount of any of the disclosed pharmaceutical compositions.

These and other aspects of the invention will be apparent upon reference to the following detailed description.

### BRIEF DESCRIPTION OF THE SEVERAL VIEWS OF THE DRAWINGS

In the drawings, the sizes and relative positions of elements in the drawings are not necessarily drawn to scale. For example, the various elements and angles are not drawn to scale, and some of these elements are arbitrarily enlarged and positioned to improve drawing legibility. Further, the particular shapes of the elements as drawn are not intended to convey any information regarding the actual shape of the particular elements, and have been selected solely for ease of recognition in the drawings.

FIG. 1 presents solubility data for amuvatinib free base in simulated intestinal fluid with various excipients.

FIG. 2 is solubility data for amuvatinib free base with different concentrations of soluplus.

FIG. 3 is a graph showing solubility of amuvatinib free base in simulated intestinal fluid with or without lipid vehicle.

FIG. 4 presents solubility data for amuvatinib HCl in supernatants of simulated intestinal fluid with lipid vehicle with or without soluplus.

FIG. 5 is a plot of dissolution data.

FIG. 6 shows results of kinetic dissolution experiments with various amuvatinib formulations versus a control formulation.

### DETAILED DESCRIPTION OF THE INVENTION

In the following description, certain specific details are set forth in order to provide a thorough understanding of various embodiments of the invention. However, one skilled in the art will understand that the invention may be practiced without these details.

Unless the context requires otherwise, throughout the present specification and claims, the word “comprise” and variations thereof, such as, “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

“Alkyl” refers to a straight or branched hydrocarbon chain radical consisting solely of carbon and hydrogen atoms, which is saturated or unsaturated (i.e., contains one or more double and/or triple bonds), having from one to twenty carbon atoms (C₁-C₂₀ alkyl), one to twelve carbon atoms (C₁-C₁₂ alkyl), preferably one to eight carbon atoms (C₁-C₈ alkyl) or one to six carbon atoms (C₁-C₆ alkyl), and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl, ethenyl, prop-1-enyl, but-1-enyl, pent-1-enyl, penta-1,4-dienyl, ethynyl, propynyl, butynyl, pentynyl, hexynyl, and the like. Unless stated otherwise specifically in the specification, an alkyl group may be optionally substituted.

“Alkylene” or “alkylene chain” refers to a straight or branched divalent hydrocarbon chain linking the rest of the molecule to a radical group, consisting solely of carbon and hydrogen, which is saturated or unsaturated (i.e., contains one or more double and/or triple bonds), and having from one to twelve carbon atoms, e.g., methylene, ethylene, propylene, n-butylene, ethenylene, propenylene, n-butenylene, propynylene, n-butynylene, and the like. The alkylene chain is attached to the rest of the molecule through a single or double bond and to the radical group through a single or double bond. The points of attachment of the alkylene chain to the rest of the molecule and to the radical group can be through one carbon or any two carbons within the chain. Unless stated otherwise specifically in the specification, an alkylene chain may be optionally substituted.

“Polyoxyalkylene” includes compounds having one of the following structures: RₐO(R_{b}O)ₙ—, Rₐ(R_{b}O)ₙ— or Rₐ(OR_{b})ₙ—, wherein Rₐ is H or alkyl, R_{b} is, at each occurrence, independently an alkylene and n is an integer greater than 1. Exemplary polyoxyalkylenes include, but are not limited to, polyethylene glycol. Unless stated otherwise specifically in the specification, an alkylene chain may be optionally substituted.

“Tocopherol” includes compounds comprising the following structure:

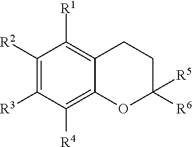

or salts thereof, wherein:

R¹, R², R³ and R⁴ are each independently H, hydroxyl, C₁-C₆ alkyl or a moiety comprising a polyoxyalkylene;

R⁵ is H or C₁-C₆ alkyl; and

R⁶ is C₁-C₂₀ alkyl.

Tocopherol includes α-, β-, γ- and δ-tocopherol, wherein alpha, beta, gamma and delta indicates the number and position of methyl groups on the chromanol ring.

“Surfactant polymer” or “surface active polymer” refers to a polymer (i.e., a compound comprising two or more repeating subunits) which lowers the surface tension between two liquids or between a liquid and a solid. Surfactant polymers are usually organic compounds that are amphiphilic, meaning they contain both hydrophobic groups and hydrophilic groups (their heads).

“Amuvatinib” refers to a compound having the following structure:

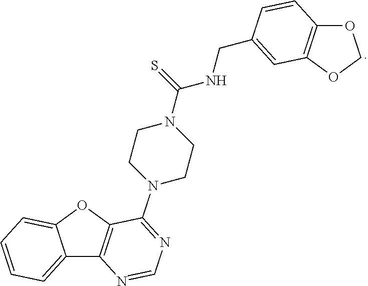 [SMILES: S=C(NCc1ccc2c(c1)OCO2)N1CCN(c2ncnc3c2oc2ccccc23)CC1]

Amuvatinib is also referred to herein as “MP470.” “MP470.HCl” or “amuvatinib HCl” refers to the HCl salt of amuvatinib.

“Soluplus” refers to polyvinyl caprolactampolyvinyl acetate-polyethylene glycol copolymer. In one embodiment the surfactant polymer Soluplus has the following structure:

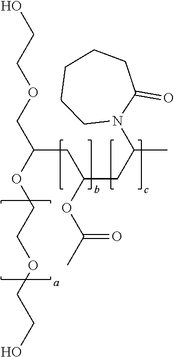

or a pharmaceutically acceptable salt thereof, wherein a, b and c are integers greater than one. In some embodiments, a, b and c are integers greater than one, and a, b and c are selected such that the surfactant polymer has an average molecular weight, as determined by gel permeation chromatography, ranging from about 90,000 g/mol to about 140,000 g/mol, for example about 118,000 g/mol (CAS Reg No. 402932-23-4). In other embodiments, a, b and c are integers greater than one, and the ratio of a to b to c (a:b:c) ranges from about 10-20 to 25-35 to 50-60, for example about 13:30:57. Soluplus is commercially available and can be prepared in a manner as known to the skilled person for example in a manner analogous to that detailed in WO 2002/018526, WO 2007/051743, WO 2007/051742 and WO 2009/013202.

“Poloxamers” include nonionic triblock copolymers comprising a central hydrophobic chain of polyoxypropylene (poly(propylene oxide)) flanked by two hydrophilic chains of polyoxyethylene (poly(ethylene oxide)).

“Prodrug” is meant to indicate a compound that may be converted under physiological conditions or by solvolysis to a biologically active compound of the invention. Thus, the term “prodrug” refers to a metabolic precursor of a compound of the invention that is pharmaceutically acceptable. A prodrug may be inactive when administered to a subject in need thereof, but is converted in vivo to an active compound of the invention. Prodrugs are typically rapidly transformed in vivo to yield the parent compound of the invention, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)). A discussion of prodrugs is provided in Higuchi, T., et al., A.C.S. Symposium Series, Vol. 14, and in Bioreversible Carriers in Drug Design, Ed. Edward B. Roche, American Pharmaceutical Association and Pergamon Press, 1987.

The term “prodrug” is also meant to include any covalently bonded carriers, which release the active compound of the invention in vivo when such prodrug is administered to a mammalian subject. Prodrugs of a compound of the invention may be prepared by modifying functional groups present in the compound of the invention in such a way that the modifications are cleaved, either in routine manipulation or in vivo, to the parent compound of the invention. Prodrugs include compounds of the invention wherein a hydroxy, amino or mercapto group is bonded to any group that, when the prodrug of the compound of the invention is administered to a mammalian subject, cleaves to form a free hydroxy, free amino or free mercapto group, respectively. Examples of prodrugs include, but are not limited to, acetate, formate and benzoate derivatives of alcohol or amide derivatives of amine functional groups in the compounds of the invention and the like.

The invention disclosed herein is also meant to encompass all pharmaceutically acceptable compounds of any of the described compounds (e.g., BCS class II or class IV drugs) being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabelled compounds could be useful to help determine or measure the effectiveness of the compounds, by characterizing, for example, the site or mode of action, or binding affinity to pharmacologically important site of action. Certain isotopically-labelled compounds of structure (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of structure (I) can generally be prepared by conventional techniques known to those skilled in the art or by processes analogous to those described in the Preparations and Examples as set out below using an appropriate isotopically-labeled reagent in place of the non-labeled reagent previously employed.

The invention disclosed herein is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the invention includes pharmaceutical compositions comprising compounds produced by a process comprising administering any of the disclosed compounds to a mammal for a period of time sufficient to yield a metabolic product thereof. Such products are typically identified by administering a radiolabelled compound of the invention in a detectable dose to an animal, such as rat, mouse, guinea pig, monkey, or to human, allowing sufficient time for metabolism to occur, and isolating its conversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

“Mammal” includes humans and both domestic animals such as laboratory animals and household pets (e.g., cats, dogs, swine, cattle, sheep, goats, horses, rabbits), and non-domestic animals such as wildlife and the like.

“Patient” includes mammals. Typically, a patient will be in need of treatment for certain disease, such as cancer or other diseases treatable with a BCS class II or class IV compound. Patient includes paediatric and adult mammals e.g. humans.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

In certain embodiments, the inventive pharmaceutical compositions include solvates of the compounds (i.e., drugs). As used herein, the term “solvate” refers to an aggregate that comprises one or more molecules of a compound of the invention with one or more molecules of solvent. The solvent may be water, in which case the solvate may be a hydrate. Alternatively, the solvent may be an organic solvent. Thus, the compounds of the present invention may exist as a hydrate, including a monohydrate, dihydrate, hemihydrate, sesquihydrate, trihydrate, tetrahydrate and the like, as well as the corresponding solvated forms. The compound of the invention may be true solvates, while in other cases, the compound of the invention may merely retain adventitious water or be a mixture of water plus some adventitious solvent.

A “pharmaceutical composition” refers to a formulation of a compound (drug) and a medium generally accepted in the art for the delivery of the biologically active compound to mammals, e.g., humans. Such a medium includes all pharmaceutically acceptable carriers, diluents or excipients therefor.

“Effective amount” or “therapeutically effective amount” refers to that amount of a compound or pharmaceutical composition of the invention which, when administered to a mammal, preferably a human, is sufficient to effect treatment, as defined below, of a disease, such as cancer, in the mammal, preferably a human. The amount of a compound or pharmaceutical composition of the invention which constitutes a “therapeutically effective amount” will vary depending on the compound or pharmaceutical composition, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest in a mammal, preferably a human, having the disease or condition of interest, and includes:

(i) preventing the disease or condition from occurring in a mammal, in particular, when such mammal is predisposed to the condition but has not yet been diagnosed as having it;

(ii) inhibiting the disease or condition, e.g., arresting its development;

(iii) relieving or alleviating the disease or condition, e.g., causing regression of the disease or condition; or

(iv) relieving or alleviating the symptoms resulting from the disease or condition, e.g., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The compounds described herein, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to pharmaceutical compositions comprising all such possible isomers, as well as their racemic and optically pure forms. Optically active (+) and (−), (R)- and (S)-, or (D)- and (L)- isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

A “tautomer” refers to a proton shift from one atom of a molecule to another atom of the same molecule. The present invention includes tautomers of any said compounds.

The term “BCS class II drug” refers to drug substances with high permeability and low solubility. A drug has high permeability when the extent of absorption in humans is determined to be >90% of an administered dose based on mass-balance pharmacokinetics studies, absolute bioavailability studies, or in comparison to an intravenous reference dose. A drug has low solubility when the highest dose strength is not soluble in <250 ml water over a pH range of 1 to 7.5, based on a shake-flask or titration method and analysed by a validated stability-indicating assay. Non-limiting examples of BCS class II and predicted BCS class II drugs include the following drugs: albendazole, amiodarone, atorvastatin, azithromycin, camptothecin, carbamazepine, carvedilol, chlorpromazine, ciprofloxacin, cisapride, clofazamine, cyclosporine, danazol, diclofenac, diflunisal, digoxin, efavirenz, erythromycin, famotidine, fenofibrate, flurbiprofen, gilbenclamide, glipizide, glyburide, griseofulvin, haloperidol, ibuprofen, indinavir, indomethacin, itraconazole, ivermectin, ketoconazole, lansoprazole, lopinavir, lovastatin, mefloquin, nalidixic acid, naproxen, nelfinavir, nevirapine, oxaprozin, phenytoin, piroxicam, praziquantel, raloxifene, rentinol palmitate, rifampin, ritonavir, saquinavir, sirolimus, spironolactone, sulfasalazine, tacrolimus, tamoxifen, terfenadine, and warfarin.

The term “BCS class IV drug” refers to drug substances with low permeability and low solubility. A drug has low permeability when the extent of absorption in humans is determined to be <90% of an administered dose, based on mass-balance pharmacokinetics studies, absolute bioavailability studies, or in comparison to an intravenous reference dose. A drug has low solubility when the highest dose strength is not soluble in <250 ml water over a pH range of 1 to 7.5, based on a shake-flask or titration method and analysed by a validated stability-indicating assay. Non-limiting examples of BCS class IV and predicted class IV drugs include the following drugs: acetazolamide, amphotericin, amphotericin B, amuvatinib, chlorothiazide, chlorthalidone, ciprofloxacin, colistin, hydrochlorothiazide, methotrexate, neomycin, nitrofurantoin, and nystatin.

Additional non-limiting examples of drugs that are predicted to be either BCS class II or BCS class IV drugs may include the following drugs: azathioprine, dapsone, furosemide, mebendazole, ofloxacin, phenazopyridine, sulfamethoxazole, and talinolol. The BCS classification system is well-known to those of ordinary skill in the art and is described in more detail in Takagi T, Ramachandran C, Bermejo M, Yamashita S, Yu L X, Amidon G L. “A provisional biopharmaceutical classification of the top 200 oral drug products in the United States, Great Britain, Spain, and Japan” Mol Pharmaceutics. 2006; 3:631-643. doi: 10.1021/mp0600182, the full disclosure of which is hereby incorporated by reference in its entirety.

A “chemotherapeutic agent” is any chemical, alone or in combination with another chemical or treatment, used to treat any disease. In certain non-limiting embodiments the chemotherapeutic agent is one or more of the following agents: a mitotic inhibitor, alkylating agent, anti-metabolite, cell cycle inhibitor, enzymes, topoisomerase inhibitor, biological response modifier, anti-hormone, antiangiogenic agent, anti-androgen, platinum coordination complex, substituted urea, methylhydrazine derivative, adrenocortical suppressant, hormone and hormone antagonist, progestin, estrogen, antiestrogen, androgen, and aromatase inhibitor. In certain non-limiting embodiments the chemotherapeutic agent is one or more of the following agents: DNA damaging agent, wherein the DNA damaging agent may be selected, for example, from the group consisting of gamma radiation; platinums, such as cisplatin, carboplatin, satraplatin, and oxaliplatin; topoisomerase I inhibitors, such as camptothecin, irinotecan, and topotecan; and topoisomerase II inhibitors, such as etoposide and teniposide.

The term “protein kinase-mediated disease” is a disease, condition, undesirable condition, or syndrome 1) that is caused or exasperated by a protein kinase, or 2) in which a protein kinase is known to play a role, or 3) that may be treated by modifying a protein kinase in any way, including but not limited to activating, inactivating, down regulating, up regulating, modifying the kinase, or modifying the localization of the kinase. Such conditions include, without limitation, cancer and other hyperproliferative disorders. In certain embodiments, the cancer is a cancer of colon, breast, stomach, prostate, pancreas, or ovarian tissue. In certain embodiments, the cancer is a cancer of the soft tissue or endocrine system. In certain embodiments the kinase mediated disease is aurora-2 kinase-mediated disease, a c-kit-mediated disease, a PDGFR-a-mediated disease, a c-ret-mediated disease or a c-met-mediated disease.

The term “Aurora-2 kinase-mediated disease” or “condition”, as used herein, means any disease or other deleterious condition in which Aurora is known to play a role. The term “Aurora-2 kinase-mediated disease” or “condition” also means those diseases or conditions that are alleviated by treatment with an Aurora-2 inhibitor or activator.

The term “C-kit-mediated disease” or “condition”, as used herein, means any disease or other deleterious condition in which C-kit is known to play a role. The term “C-kit kinase-mediated disease” or “condition” also means those diseases or conditions that are alleviated by treatment with a C-kit inhibitor or activator.

The term “PDGFR-a-mediated disease” or “condition”, as used herein, means any disease or other deleterious condition in which PDGFR is known to play a role. The term “PDGFR kinase-mediated disease” or “condition” also means those diseases or conditions that are alleviated by treatment with a PDGFR inhibitor or activator.

The term “C-ret-mediated disease” or “condition”, as used herein, means any disease or other deleterious condition in which C-ret is known to play a role. The term “C-ret kinase-mediated disease” or “condition” also means those diseases or conditions that are alleviated by treatment with a C-ret inhibitor or activator.

The term “C-met-mediated disease” or “condition”, as used herein, means any disease or other deleterious condition in which C-met is known to play a role. The term “C-met kinase-mediated disease” or “condition” also means those diseases or conditions that are alleviated by treatment with a C-met inhibitor or activator.

The term ‘average molecular weight’ is used in the art to indicate that the average molecular weight of polymeric materials can be measured in a number of different manners, including peak average molecular weight (Mp), number average molecular weight (Mn), and weight average molecular weight (Mw). In particular embodiments for Soluplus the average molecular weight is the weight average molecular weight (Mw).

As noted above, in one embodiment of the present invention, a pharmaceutical composition is provided. Applicants have unexpectedly discovered that the bioavailability of BCS class II and class IV drugs, such as amuvatinib, is synergistically increased by the combination of a lipid vehicle and a surfactant polymer. In certain embodiments, the surfactant polymer is Soluplus™ which heretofore has only been described for use in hot melt extrusion formulations, and its surprising synergy when combined with a lipid vehicle is unexpected based on any of its prior-described uses. The inventive compositions increase the bioavailability of poorly soluble drugs, thus providing an improved exposure of the compound. This enables capsules of higher strengths to be prepared thus reducing the total dosage and pill requirement, resulting in higher patient compliance and reduced cost of goods.

In one aspect, the present invention provides a pharmaceutical composition comprising:

a) a BCS class II or class IV drug, or a stereoisomer, tautomer, pharmaceutically acceptable salt or prodrug thereof;

b) a surfactant polymer, or a pharmaceutically acceptable salt thereof;

c) a tocopherol, or a pharmaceutically acceptable salt thereof; and

d) a fatty acid, a fatty acid ester, or a pharmaceutically acceptable salt thereof.

In more particular embodiments, the drug is amuvatinib, or a pharmaceutical salt thereof, and the pharmaceutical composition comprises:

a) amuvatinib, or a stereoisomer, tautomer, pharmaceutically acceptable salt or prodrug thereof;

b) a surfactant polymer, or a pharmaceutically acceptable salt thereof;

c) a tocopherol, or a pharmaceutically acceptable salt thereof; and

d) a fatty acid, a fatty acid ester, or a pharmaceutically acceptable salt thereof.

In any of the foregoing embodiments, the surfactant polymer comprises a polyoxyalkylene a polysaccharide, or a polyvinylpyrrolidone. For example, in certain embodiments, the surfactant polymer comprises a polyoxyalkylene. In other embodiments, the polyoxyalkylene is a graft copolymer, such as a graft copolymer which comprises a polyoxyalkylene, a polyvinyl lactam and a polyvinyl ester. In even more specific embodiments, the graft copolymer is a polyvinyl caprolactam-polyvinyl acetate-polyethylene glycol copolymer.

In some more of the foregoing embodiments, the surfactant copolymer has a molecular weight ranging from about 90,000 g/mol to about 140,000 g/mol. In still more embodiments, the surfactant polymer has the following structure:

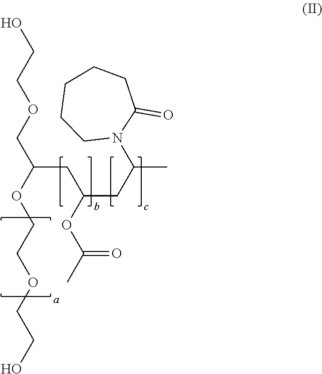

or a pharmaceutically acceptable salt thereof, wherein a, b and c are integers greater than one and a, b and c are selected such that the surfactant polymer has an average molecular weight, as determined by gel permeation chromatography, ranging from about 90,000 g/mol to about 140,000 g/mol, for example about 118,000 g/mol in some embodiments. In other embodiments, a, b and c are integers greater than one, and a, b, and c are selected such that the ratio of a to b to c (a:b:c) ranges from about 10-20 to 25-35 to 50-60, for example about 13:30:57.

In some different embodiments of the above, the polyoxyalkylene is a poloxamer. For example, in some embodiment the poloxamer has a molecular weight ranging from about 1,000 g/mol to about 4,000 g/mol, such as from about 1,800 g/mol to about 3,600 g/mol. In some more embodiments, the poloxamer comprises from about 60% to about 90% by weight of polyethylene oxide, for example from about 70% to about 80% by weight of polyethylene oxide. In certain more specific embodiments, the poloxamer is Pluronic F68, Pluorinic F87, Pluronic F108 or Pluronic F127 (which are commercially available poloxamers well-known in the art).

In still other different embodiments, the polyoxyalkylene is a polyethylene glycol. For example, in certain embodiments the polyethylene glycol is PEG3350.

In some more different embodiments, the surfactant polymer comprises a polysaccharide. For example, the polysaccharide may be a cellulose, such as HPMC, HPMCAS-LG, HPMCAS-MG, HPMCAS-HG or HPMC-P (which are commercially available and well-known in the art). In other embodiments, the cellulose is hydroxypropyl methylcellulose acetate succinate. In still more embodiments, the polysaccharide is a cyclic polysaccharide, such as a cyclodextrin like β-cyclodextrin, HPBCD or MCD.

In still other different embodiments, the surfactant polymer comprises a polyvinylpyrrolidone. In some of these embodiments, the surfactant polymer is PVP-VA64, PVP-K60 or PVP-K30.

As noted above, the solubility of amuvatinib and other BCS class II and class IV drugs is unexpectedly increased by selection of an appropriate lipid vehicle. In some embodiments, the lipid vehicle includes a tocopherol and a fatty acid or fatty acid ester (and pharmaceutically acceptable salts of the foregoing).

In any of the foregoing embodiments, the tocopherol is a α, β, γ or δ tocopherol. In some of these embodiments, the tocopherol further comprises a polyalkylene oxide moiety. For example, the polyalkylene oxide moiety may be a polyethylene glycol. In some more specific embodiments, the tocopherol is D-alpha tocopherol polyethylene glycol 1000 succinate.

One particular tocopherol is the D-α-tocopherol acid, D-α-tocopheryl polyethylene glycol succinate (Vitamin E TPGS, or simply TPGS, CAS Reg No. 9002-96-4) a water-soluble derivative of natural Vitamin E, which is formed by esterification of Vitamin E succinate with polyethylene glycol (PEG). Typically, the molecular weight of TPGS with PEG1000 segment is 1513. In one embodiment the tocopherol is D-α-tocopheryl polyethylene glycol 1000 succinate. TPGS has an amphiphilic structure of lipophilic alkyl tail and hydrophilic polar head with a hydrophile-lipophile balance (HLB) value of 13.2 and a critical micelle concentration (CMC) of 0.02% w/w. US FDA has approved TPGS as a safe pharmaceutical adjuvant used in drug formulation. TPGS is commercially available and has the following chemical structure:

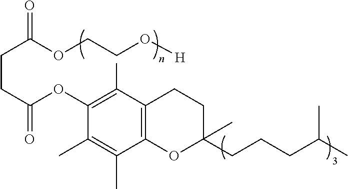

In some embodiments of the above structure, n is an integer from about 10 to about 100, from about 25 to about 75 or from about 55 to about 65. For example, in certain embodiments n is selected such that TPGS has a molecular weight of about 1513 g/mol.

In any of the foregoing pharmaceutical compositions, the pharmaceutical composition comprises a fatty acid ester for example a polyalkylene oxide fatty acid ester (i.e., an ester of a polyalkylene oxide and a fatty acid), or pharmaceutically acceptable salt thereof. For example, the polyalkylene oxide fatty acid ester may be an ester of a long chain fatty acid. In certain embodiments, the long chain fatty acid is a C-18 fatty acid. For example, in some embodiments the long chain fatty acid is stearic acid or ricinoleic acid or hydrogenated or hydroxylated derivatives thereof. In some even more specific embodiments, the fatty acid or fatty acid ester is glycerol polyethylene glycol 12-hydroxystearate (also known as Polyoxyl castor oil, PEG-40 solid Hydrogenated Castor Oil or Cremophor RH40), polyoxyl 15 12-hydroxystearate (Solutol HS 15) or PEG 20 stearate (Lipopeg 10-S). Cremophor RH40 is also known as polyoxyl 40 hydrogenated castor Oil (USP) or macrogolglycerol hydroxystearate (Ph. Eur.) and is commercially available.

The ratio of drug to surfactant polymer is selected for optimal solubility. In any of the foregoing embodiments, a ratio amuvatinib (or other BCS class II or class IV drug) to the surfactant polymer ranges from about 1:0.1 to about 0.1:1. In other embodiments, the ratio ranges from about 2:0.5 to about 2:4. In still more embodiments, the ratio is about 2:1 to about 3:1.

In still more embodiments, the ratio of drug (e.g., amuvatinib or other BCS class II or class IV drug) to total surfactant (i.e., tocopherol+surfactant polymer) ranges from about 1:2 to about 1:10, from about 1:3 to about 1:9, from about 1:4 to about 1:8. For example in some embodiments the ratio is about 1:7, for example about 6.6.

In still more embodiments, the ratio fatty acid (or ester thereof) to total surfactant (surfactant polymer+tocopherol) ranges from about 1:1 to about 1:5, for example about 1:1 to about 1:4 or about 1:2 to about 1:3.

In still more embodiments of the foregoing, the ratio of ratio fatty acid (or ester thereof) to tocopherol ranges from about 10:1 to about 2:1, for example about 8:1 to about 2:1, about 6:1 to about 2:1 or about 5:1 to about 3:1. In some embodiments, the ratio is about 4:1.

In still more embodiments, the ratio of drug to surfactant polymer to tocopherol ranges from 1:1:1 to about 1:6:1

In other embodiments of the above, the BCS class II or class IV drug (e.g., amuvatinib) is present in the pharmaceutical composition in a mass percentage ranging from about 1% to about 10%, for example from about 4% to about 8%.

In some other of the foregoing embodiments, the pharmaceutical composition is formulated as a capsule. In some other of the foregoing embodiments, the pharmaceutical composition is a solid dispursion. A solid dispersion in selected polymers can be prepared by solvent evaporation, spray drying, lyophilization, or hot melt dispersion.

In still more embodiments of any of the foregoing, the pharmaceutical composition further comprises one or more other chemotherapeutic agents. For example, in some embodiments the chemotherapeutic agent is selected from mitotic inhibitors, alkylating agents, anti-metabolites, cell cycle inhibitors, enzymes, topoisomerase inhibitors such as CAMPTOSAR (irinotecan), biological response modifiers, anti-hormones, antiangiogenic agents such as MMP-2, MMP-9 and COX-2 inhibitors, anti-androgens, platinum coordination complexes (cisplatin, etc.), substituted ureas such as hydroxyurea, methylhydrazine derivatives e.g., procarbazine, adrenocortical suppressants e.g., mitotane or aminoglutethimide, hormone and hormone antagonists such as the adrenocorticosteriods (e.g., prednisone), progestins (e.g., hydroxyprogesterone caproate), estrogens (e.g., diethylstilbesterol), antiestrogens such as tamoxifen, androgens e.g., testosterone propionate, and aromatase inhibitors such as anastrozole, and exemestane. In other embodiments, the chemotherapeutic agent is a DNA-damaging agent, wherein the DNA damaging agent may be selected, for example, from the group consisting of gamma radiation; platinums, such as cisplatin, carboplatin, satraplatin, and oxaliplatin; topoisomerase I inhibitors, such as camptothecin, irinotecan, and topotecan; and topoisomerase II inhibitors, such as etoposide and teniposide.

Examples of alkylating agents that the above method can be carried out in combination with include, without limitation, fluorouracil (5-FU) alone or in further combination with leukovorin; other pyrimidine analogs such as UFT, capecitabine, gemcitabine and cytarabine, the alkyl sulfonates, e.g., busulfan (used in the treatment of chronic granulocytic leukemia), improsulfan and piposulfan; aziridines, e.g., benzodepa, carboquone, meturedepa and uredepa; ethyleneimines and methylmelamines, e.g., altretamine, triethylenemelamine, triethylenephosphoramide, triethylenethiophosphoramide and trimethylolmelamine; and the nitrogen mustards, e.g., chlorambucil (used in the treatment of chronic lymphocytic leukemia, primary macroglobulinemia and non-Hodgkin's lymphoma), cyclophosphamide (used in the treatment of Hodgkin's disease, multiple myeloma, neuroblastoma, breast cancer, ovarian cancer, lung cancer, Wilm's tumor and rhabdomyosarcoma), estramustine, ifosfamide, novembrichin, prednimustine and uracil mustard (used in the treatment of primary thrombocytosis, non-Hodgkin's lymphoma, Hodgkin's disease and ovarian cancer); and triazines, e.g., dacarbazine (used in the treatment of soft tissue sarcoma).

Examples of antimetabolite chemotherapeutic agents that the above method can be carried out in combination with include, without limitation, folic acid analogs, e.g., methotrexate (used in the treatment of acute lymphocytic leukemia, choriocarcinoma, mycosis fungiodes, breast cancer, head and neck cancer and osteogenic sarcoma) and pteropterin; and the purine analogs such as mercaptopurine and thioguanine which find use in the treatment of acute granulocytic, acute lymphocytic and chronic granulocytic leukemias.

Examples of natural product-based chemotherapeutic agents that the above method can be carried out in combination with include, without limitation, the vinca alkaloids, e.g., vinblastine (used in the treatment of breast and testicular cancer), vincristine and vindesine; the epipodophyllotoxins, e.g., etoposide and teniposide, both of which are useful in the treatment of testicular cancer and Kaposi's sarcoma; the antibiotic chemotherapeutic agents, e.g., daunorubicin, doxorubicin, epirubicin, mitomycin (used to treat stomach, cervix, colon, breast, bladder and pancreatic cancer), dactinomycin, temozolomide, plicamycin, bleomycin (used in the treatment of skin, esophagus and genitourinary tract cancer); and the enzymatic chemotherapeutic agents such as L-asparaginase.

Examples of chemotherapeutic agents include signal transduction inhibitors, such as agents that can inhibit EGFR (epidermal growth factor receptor) responses, such as EGFR antibodies, EGF antibodies, and molecules that are EGFR inhibitors; VEGF (vascular endothelial growth factor) inhibitors; and erbB2 receptor inhibitors, such as organic molecules or antibodies that bind to the erbB2 receptor, such as HERCEPTIN (Genentech, Inc., South San Francisco, Calif.). EGFR inhibitors are described in, for example in WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998), and such substances can be used in the present invention as described herein, for example erlotinib and imatinib.

EGFR-inhibiting agents include, but are not limited to, the monoclonal antibodies C225 and anti-EGFR 22Mab (ImClone Systems, Inc., New York, N.Y.), the compounds ZD-1839 (AstraZeneca), BIBX-1382 (Boehringer Ingelheim), MDX-447 (Medarex Inc., Annandale, N.J.), and OLX-103 (Merck & Co., Whitehouse Station, N.J.), and EGF fusion toxin (Seragen Inc., Hopkinton, Mass.).

Examples of chemotherapeutic agents include agents capable of enhancing antitumor immune responses, such as CTLA4 (cytotoxic lymphocite antigen 4) antibodies, and other agents capable of blocking CTLA4; and anti-proliferative agents such as other farnesyl protein transferase inhibitors, for example the farnesyl protein transferase inhibitors.

The above method can be also be carried out in combination with radiation therapy, wherein the amount of a compound in combination with the radiation therapy is effective in treating the diseases described herein for example cancer.

In some of the foregoing pharmaceutical compositions, the pharmaceutical composition comprises a BCS class II drug, or a stereoisomer, tautomer, pharmaceutically acceptable salt or prodrug thereof. In some of these embodiments, the BCS class II drug is danazol, fenofibrate, itraconazole, amiodarone, atorvastatin, azithromycin, carbamazepine, camptothecin, carvedilol, chlorpromazine, cisapride, ciprofloxacin, cyclosporine, dapsone, diclofenac, diflunisal, digoxin, erythromycin, flurbiprofen, famotidine, glipizide, gilbenclamide, glyburide, griseofulvin, ibuprofen, indinavir, indomethacin, itraconazole, ketoconazole, lansoprazolel, lovastatin, mebendazole, naproxen, nelfinavir, ofloxacin, oxaprozin, phenazopyridine, phenytoin, piroxicam, praziquantel, raloxifene, ritonavir, rentinol palmitate, saquinavir, sirolimus, spironolactone, sulfasalazine, tacrolimus, talinolol, tamoxifen or terfenadine.

In some different embodiments of the foregoing, the pharmaceutical composition comprises a BCS class IV drug, or a stereoisomer, tautomer, pharmaceutically acceptable salt or prodrug thereof. In some of these embodiments the BCS class IV drug is amuvatinib, amphotericin, chlorthalidone, chlorothiazide, colistin, furosemide, hydrochlorothiazide, methotrexate, nitrofurantoin, neomycin or paracetamol.

In addition to pharmaceutical compositions, embodiments of the present invention include kits. Such kits are advantageous in that the active drug and the carrier can be stored in separate packages until use, thus potentially increasing the shelf life of the active compound. In certain embodiments, a kit comprises a solid dispersion and a vehicle in separate packages, wherein:

the solid dispersion comprises a BCS class II or class IV drug (e.g., amuvatinib), or a stereoisomer, tautomer, pharmaceutically acceptable salt or prodrug thereof, and a surfactant polymer, or pharmaceutically acceptable salt thereof; and

the vehicle comprises a tocopherol, or pharmaceutically acceptable salt thereof, and a fatty acid, a fatty acid ester, or pharmaceutically acceptable salt thereof.

In this regard, the surfactant polymer, tocopherol and fatty acid (or ester thereof) are as defined in any of the foregoing embodiments.

In some embodiments, the kit further comprises instructions for admixing the solid dispersion with the vehicle prior to administration to a mammal in need of treatment with the BCS class II or class IV drug (e.g., amuvatinib).

In some embodiments, the solid dispersion comprises a BCS class IV drug.

In other more specific embodiments, the solid dispersion comprises amuvatinib or amuvatinib hydrochloride.

In other embodiments, the kits comprise an active drug and a vehicle in separate packages, wherein:

the active drug is a BCS class II or class IV drug, such as amuvatinib, or a stereoisomer, tautomer, pharmaceutically acceptable salt or prodrug thereof; and

the vehicle comprises a surfactant polymer, or pharmaceutically acceptable salt thereof, a tocopherol, or pharmaceutically acceptable salt thereof, and a fatty acid or fatty acid ester, or pharmaceutically acceptable salt thereof.

The surfactant polymer, tocopherol and fatty acid (or ester thereof) are again as defined as in any of the foregoing embodiments.

In some embodiments of the foregoing kit, the kit further comprises instructions for admixing the active drug with the vehicle prior to administration to a mammal in need of treatment with the active drug.

In certain embodiments, the active drug is a BCS class IV drug. In more specific embodiments the active drug is amuvatinib or amuvatinib hydrochloride.

Other embodiments of the present invention includes a method for increasing the bioavailability of a BCS class II or class IV drug, the method comprising preparing a pharmaceutical composition comprising:

the BCS class II or class IV drug, or a stereoisomer, tautomer, pharmaceutically acceptable salt or prodrug thereof;

a surfactant polymer, or a pharmaceutically acceptable salt thereof;

a tocopherol, or a pharmaceutically acceptable salt thereof; and

a fatty acid or fatty acid ester, or a pharmaceutically acceptable salt thereof.

In some specific embodiments, the drug is amuvatinib or amuvatinib hydrochloride.

The surfactant polymer, tocopherol and fatty acid (or ester thereof) in the foregoing method are as defined in any of the foregoing embodiments.

In some embodiments, the method comprises administering the pharmaceutical composition to a mammal in need of treatment with the BCS class II or class IV drug.

In still other embodiments, the invention provides a method for the treatment of a protein kinase-mediated disease, the method comprising administering to a subject in need thereof a therapeutically effective amount of any of the foregoing pharmaceutical compositions (e.g., compositions comprising amuvatinib or amuvatinib hydrochloride). In some embodiments, the protein kinase-mediated disease is an aurora-2 kinase-mediated disease, a c-kit-mediated disease, a PDGFR-a-mediated disease, a c-ret-mediated disease or a c-met-mediated disease. In other embodiments, the protein-kinase mediated disease is cancer. In other embodiments, the invention provides a method for the treatment of a cancer. In a further embodiment, the invention provides a pharmaceutical composition for use in the prophylaxis or treatment (e.g. reducing or allieviating) of a cancer. In a further embodiment, the invention provides a pharmaceutical composition for use in the prophylaxis or treatment (e.g. reducing or allieviating) a protein kinase-mediated disease.

As mentioned above, the compounds and compositions of the invention will find utility in a broad range of diseases and conditions mediated by protein kinases, including diseases and conditions mediated by aurora-2 kinase, c-kit and/or PDGFR-a. Such diseases may include by way of example and not limitation, cancers such as lung cancer, NSCLC (non small cell lung cancer), oat-cell cancer, bone cancer, pancreatic cancer, skin cancer, dermatofibrosarcoma protuberans, cancer of the head and neck, cutaneous or intraocular melanoma, uterine cancer, ovarian cancer, colo-rectal cancer, cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's Disease, hepatocellular cancer, cancer of the esophagus, cancer of the small intestine, cancer of the endocrine system (e.g., cancer of the thyroid, pancreas, parathyroid or adrenal glands), sarcomas of soft tissues, cancer of the urethra, cancer of the penis, prostate cancer (particularly hormone-refractory), chronic or acute leukemia, solid tumors of childhood, hypereosinophilia, lymphocytic lymphomas, cancer of the bladder, cancer of the kidney or ureter (e.g., renal cell carcinoma, carcinoma of the renal pelvis), pediatric malignancy, neoplasms of the central nervous system (e.g., primary CNS lymphoma, spinal axis tumors, medulloblastoma, brain stem gliomas or pituitary adenomas), Barrett's esophagus (pre-malignant syndrome), neoplastic cutaneous disease, psoriasis, mycoses fungoides, and benign prostatic hypertrophy, diabetes related diseases such as diabetic retinopathy, retinal ischemia, and retinal neovascularization, hepatic cirrhosis, angiogenesis, cardiovascular disease such as atherosclerosis, immunological disease such as autoimmune disease and renal disease.

Specific types of cancers or malignant tumors, either primary or secondary, that can be treated using this invention include breast cancer, skin cancer, bone cancer, prostate cancer, liver cancer, lung cancer, brain cancer, cancer of the larynx, gall bladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuromas, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas, malignant melanomas, epidermoid carcinomas, and other carcinomas and sarcomas.

Hematologic disorders include abnormal growth of blood cells which can lead to dysplastic changes in blood cells and hematologic malignancies such as various leukemias. Examples of hematologic disorders include but are not limited to acute myeloid leukemia, acute promyelocytic leukemia, acute lymphoblastic leukemia, chronic myelogenous leukemia, the myelodysplastic syndromes, and sickle cell anemia.

In one embodiment the cancer is a cancer of the pancreas, breast, ovary or colon. In still more embodiments, the cancer is a cancer of the soft tissues. In yet more embodiments, the cancer is a cancer of the endocrine system. Certain indications for treatment with amuvatinib include, but are not limited to glioblastoma multiforme, ovarian cancer, gastro intestinal stromal tumors, non-small cell lung cancer, or medullary or papillary thyroid carcinoma.

In one embodiment the cancer is a cancer of the lung. In one embodiment the cancer is non-small cell lung cancer. In one embodiment the cancer is small cell lung cancer (also known as oat cell cancer).

In one embodiment the cancer is a sarcoma. In one embodiment the sarcoma is gastrointestinal stromal tumour (GIST). In one embodiment the cancer is a cancer of the stomach or digestive system. In one embodiment the cancer of the stomach or digestive system is gastrointestinal stromal tumour (GIST).

In one embodiment the cancer is a cancer of the prostate.

In still other embodiments, the invention provides use of any of the foregoing pharmaceutical compositions (e.g., compositions comprising amuvatinib or amuvatinib hydrochloride) for treatment of a protein kinase-mediated disease or cancer. In some embodiments, the protein kinase-mediated disease is an aurora-2 kinase-mediated disease, a c-kit-mediated disease, a PDGFR-a-mediated disease, a c-ret-mediated disease or a c-met-mediated disease. In other embodiments, the protein-kinase mediated disease is cancer. In other embodiments, the cancer is a cancer of the pancreas, breast, ovary or colon. In still more embodiments, the cancer is a cancer of the soft tissues. In yet more embodiments, the cancer is a cancer of the endocrine system.

In other embodiments, the present invention provides use of a BCS class II or class IV drug for preparation of a pharmaceutical composition comprising:

a) a surfactant polymer, or a pharmaceutically acceptable salt thereof;

b) a tocopherol, or a pharmaceutically acceptable salt thereof; and

c) a fatty acid, a fatty acid ester, or a pharmaceutically acceptable salt thereof, wherein the surfactant polymer, tocopherol and fatty acid (or ester thereof) are again as defined as in any of the foregoing embodiments. In some of these embodiments, the BCS class II or class IV drug is amuvatinib or amuvatinib hydrochloride.

In further embodiments of the invention there is provided a process for the production of the pharmaceutical composition described herein.

Pharmaceutical compositions of the present invention comprise a compound (e.g., a BCS class II or class IV drug such as amuvatinib) and a pharmaceutically acceptable carrier, diluent or excipient. The compound is present in the pharmaceutical composition in an amount which is effective to treat a particular disease or condition of interest—such as cancer, and preferably with acceptable toxicity to the patient. Activity of the pharmaceutical compositions can be determined by one skilled in the art. Appropriate concentrations and dosages can be readily determined by one skilled in the art.

Administration of the pharmaceutical compositions, can be carried out via any of the accepted modes of administration of agents for serving similar utilities. The pharmaceutical compositions of the invention can be prepared by combining a compound of the invention with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Pharmaceutical compositions of the invention are formulated so as to allow the active ingredients contained therein to be bioavailable upon administration of the pharmaceutical composition to a patient. Pharmaceutical compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The pharmaceutical composition to be administered will, in any event, contain a therapeutically effective amount of a compound of the invention, or a pharmaceutically acceptable salt thereof, for treatment of a disease or condition of interest in accordance with the teachings of this invention.

A pharmaceutical composition of the invention may be in the form of a solid or liquid. In one aspect, the carrier(s) are particulate, so that the pharmaceutical compositions are, for example, in tablet or powder form. The carrier(s) may be liquid, with the pharmaceutical compositions being, for example, an oral syrup, injectable liquid or an aerosol, which is useful in, for example, inhalatory administration.

When intended for oral administration, the pharmaceutical composition is preferably in either solid or liquid form, where semi-solid, semi-liquid, suspension and gel forms are included within the forms considered herein as either solid or liquid.

As a solid pharmaceutical composition for oral administration, the pharmaceutical composition may be formulated into a powder, granule, compressed tablet, pill, capsule, chewing gum, wafer or the like form. Such a solid pharmaceutical composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred pharmaceutical composition contain, in addition to the present compounds, one or more of a sweetening agent, preservatives, dye/colorant and flavor enhancer. In a pharmaceutical composition intended to be administered by injection, one or more of a surfactant, preservative, wetting agent, dispersing agent, suspending agent, buffer, stabilizer and isotonic agent may be included.

The liquid pharmaceutical compositions of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of the invention intended for either parenteral or oral administration should contain an amount of an active compound (i.e., drug) of the invention such that a suitable dosage will be obtained.

The pharmaceutical composition of the invention may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the pharmaceutical composition may include a transdermal patch or iontophoresis device.

The pharmaceutical composition of the invention may be intended for rectal administration, in the form, for example, of a suppository, which will melt in the rectum and release the drug. The pharmaceutical composition for rectal administration may contain an oleaginous base as a suitable nonirritating excipient. Such bases include, without limitation, lanolin, cocoa butter and polyethylene glycol.

The pharmaceutical composition of the invention may include various materials, which modify the physical form of a solid or liquid dosage unit. For example, the pharmaceutical composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the invention in solid or liquid form may include an agent that binds to the compound of the invention and thereby assists in the delivery of the compound. Suitable agents that may act in this capacity include a monoclonal or polyclonal antibody, a protein or a liposome.

The pharmaceutical composition of the invention may comprise dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions of the invention may be prepared by methodology well known in the pharmaceutical art. For example, a pharmaceutical composition intended to be administered by injection can be prepared by combining an active compound with sterile, distilled water so as to form a solution. A surfactant may be added to facilitate the formation of a homogeneous solution or suspension. Surfactants are compounds that non-covalently interact with the active compound so as to facilitate dissolution or homogeneous suspension of the compound in the aqueous delivery system.

The pharmaceutical compositions of the invention are typically administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy.

Pharmaceutical compositions of the invention may also be administered simultaneously with, prior to, or after administration of one or more other therapeutic agents. Such combination therapy includes administration of a single pharmaceutical dosage of the pharmaceutical composition of the invention and one or more additional active agents, as well as administration of the pharmaceutical composition of the invention and each additional active agent in its own separate pharmaceutical dosage formulation. For example, a pharmaceutical composition of the invention and the other active agent can be administered to the patient together in a single oral dosage pharmaceutical composition such as a tablet or capsule, or each agent administered in separate oral dosage formulations. Where separate dosage formulations are used, the pharmaceutical compositions of the invention and one or more additional active agents can be administered at essentially the same time, i.e., concurrently, or at separately staggered times, i.e., sequentially; combination therapy is understood to include all these regimens.

Furthermore, all compounds described herein which exist in free base or acid form can be converted to their pharmaceutically acceptable salts by treatment with the appropriate inorganic or organic base or acid by methods known to one skilled in the art. Salts of the compounds of the invention can be converted to their free base or acid form by standard techniques.

Amuvatinib can be prepared according to methods known in the art. For example, U.S. Pat. No. 7,326,713, which is hereby incorporated by reference in its entirety for all purposes, describes methods for preparation of amuvatinib and its pharmaceutically acceptable salts. Methods for preparation of other BCS class II and class IV drugs are known in the art.

The following examples are provided for purposes of illustration, not limitation.

### EXAMPLES

### Example 1

### Determination of MP470 Solubility

In a typical procedure, solubility of MP470 free base and MP470.HCl in various lipids, surfactants, and other solvents were measured and summarized in Table 1. The solubility test was performed by incubating the compound solid in testing solvents for more than 24 hours at room temperature. The suspensions were filtered through 0.2 or 0.45 microm filters prior to HPLC analysis. Some filtrates were diluted with MeOH prior to the HPLC analysis. The MP470 concentration in the supernatant was analyzed using the short HPLC method outlined in Table 2 developed with the Agilent rapid resolution system.

MP470 free base shows maximum solubility in NMP (˜300 mg/mL) and DMA (>240 mg/mL), while MP470.HCl has maximum solubility in NMP (40-80 mg/mL) and Benzyl alcohol (48 mg/mL). Only several GRAS solvents tested can dissolve MP470 free base or MP470.HCl in 10-20 mg/mL, but far below 50 mg/mL. Accordingly, amuvatinib is poorly soluble.

TABLE 1
Solubility of MP470 free base and MP470.HCl in organic solvents

| | Solubility (mg/mL) | | | Solubility (mg/mL) | |
|---|---|---|---|---|---|
| Solvent | MP470 FB | MP470.HCl | Solvent | MP470 FB | MP470.HCl |
| Acetic acid | 2.9 | 3.0 | Acetone | 2.6 | 0.2 |
| Anisole | 1.8 | | Benzyl alcohol | 63 | 48 |
| Benzyl acetate | | 0.4 | Canola oil | | 0.01 |
| Castor oil | | 0.2 | Capryol 90 | | 0.1 |
| Clove oil | | 6.7 | Cremophor EL | | 7 |
| Corn oil | | 0.01 | Dichloromethane | | 0.4 |
| DMA | >240 | 13 | DMF | | 10 |
| DMSO | ~130 | 18 | Ethanol | 0.3 | 0.4 |
| Ethyl acetate | | 0.05 | Ethyl cinnamate | 3.9 | |
| Glycerin | 0.4 | 4 | Labrasol | 11 | 3 |
| Labrafac CC | | 0.05 | Labrafil M1944CS | | 0.3 |
| Lauroglycol 90 | | 0.06 | Linoleic acid | 0.02 | |
| Maisine 35-1 | | 1.4 | Methanol | 0.5 | 3 |
| Miglyol 812N | | 0.03 | Miglyol 829 | | 0.1 |
| Miglyol 840 | | 0.04 | NMP | 300-360 | 40-80 |
| Oleic acid | 0.1 | | Peceol | | 0.2 |
| PEG400 | 14 | 2 | Peanut oil | | 0.01 |
| Propylene | 2.9 | 2.9 | Propylene glycol | 0.4 | 4 |
| carbonate | | | | | |
| Phosal 50 PG | 0.5 | 15 | Sesame oil | | 0.03 |
| Solutol HS 15 | | 6** | Triacetin | 1.0 | 0.8 |
| Tocopherol | | 0.8 | TPGS | 10 | 7** |
| Tween 20 | 8 | 17 | Tween 80 | 6.6 | 4.5 |

**The solubility was estimated by diluting the mixture in water 1/100 and then assaying the dilution.

TABLE 2
A Short HPLC Method and Parameters for MP470 Assay Analysis

| Parameter | Value | |
|---|---|---|
| Flow rate | 1.0 mL/min | |
| Stop time | 7 min | |
| Mobile phase A | 0.1% TFA in Water | |
| Mobile phase B | 0.1% TFA in Acetonitrile | |
| | Time | % B |
| Gradient table | 0 | 10 |
| | 3 | 70 |
| | 5 | 100 |
| | 6 | 100 |
| | 6.30 | 10 |
| | 7 | 10 |

| | |
|---|---|
| Injection volume | 3 μL |
| Column temp. | 30° C. |
| Auto sampler temp. | 5° C. (for this study auto sampler temperature is |
| | 37° C.) |
| UV detection | 242 nm with Bw: 16, Ref: 360 nm, Bw: 100 |
| Column | Zorbax Eclipse XDB C18 Rapid Resolution HT, |
| | 4.6 × 50 mm, 1.8 μm, |
| | PN: 927975-902, SN: USZF004294, lot B08047 |
| Sample diluent | Methanol |

### Example 2

### Preparation of Pharmaceutical Composition

In a typical procedure, 20 to 30 grams of pharmaceutical compositions were prepared for formulations described in Table 3 and Table 4 by the following steps. MP470.HCl is used as the active molecule in these examples as a non-limiting example. The vehicle was prepared by first melting the required amount of Vitamin E TPGS and Cremophor RH40 at 70° C., and then Soluplus was dissolved in the mixture at 70° C. with continuously stirring for 2 hr. The temperature of the vehicle was then lowered to 50° C., and MP470.HCl, the active pharmaceutical ingredient (“API”), was added to the vehicle. The mixture was homogenized at 5000-10000 rpm for 5-8 min, and then stirred with a magnetic stirring bar at 50° C. for 12-15 min. The capsules were filled using a positive-displacement pipette while stirring. The filled capsules were place on ice, and stored at 2-8° C.

In another typical procedure, amounts of pharmaceutical compositions appropriate for manufacture were prepared for the formulations in Table 3 and Table 4 by the following steps. MP470.HCl is used as the active molecule in these examples as a non-limiting example. The vehicle was prepared by preheating the required amount of Vitamin E TPGS, Cremophor RH40, and Soluplus at 70° C.±5° C. The Vitamin E TPGS, Cremophor RH40, and Soluplus was mixed and stirred to ensure a clear homogeneous solution was achieved. The vehicle was then cooled to 50° C.±5° C. and the MP470.HCl was added. This formulation was stirred at 50° C.±5° C. under nitrogen until homogeneity was reached. The formulation was encapsulate in size 0 capsules, and the encapsulated capsules were sealed. The capsules were sorted by QC sorting and place at 2-8° C. The capsules were stored at 2-8° C.

TABLE 3
Composition of the Enhanced Lipid-based Suspension Formulation of
MP470 (50 mg/g)

| | | Wt. (mg) for each |
|---|---|---|
| Component | Wt. (mg) | capsule |
| MP470.HCl | 5.4 | 32 |
| Soluplus | 2.5 | 15 |
| Vitamin E TPGS | 18.4 | 111 |
| Cremophor RH40 | 73.7 | 442 |
| Total Wt. (mg) | 100.0 | 600 |

TABLE 4
Composition of the Enhanced Lipid-based Suspension Formulation of
MP470 (75 mg/g)

| | | Wt. (mg) for each |
|---|---|---|
| Component | Wt. (mg) | capsule |
| MP470.HCl | 8.2 | 49 |
| Soluplus | 3.8 | 23 |
| Vitamin E TPGS | 17.6 | 106 |
| Cremophor RH40 | 70.4 | 422 |
| Total Wt. (mg) | 100.0 | 600 |

### Example 3

### Analysis of Active Ingredient Release from Various Excipients in Simulated Intestinal Fluid

A collection of 17 polymers of various kinds (Table 5) were tested for their capability to inhibit MP470 precipitation in simulated intestinal fluid (SIF). In a typical procedure, MP470 FB solution in NMP solvent (200 mg/mL) was diluted (1/1000) into each of the polymer solutions pre-warmed at 37° C. The mix was incubated at 37° C. for a period of time (2 hr, 4 hr) prior to aliquoting out a sample for analysis of MP470 FB concentration in the supernatant. The MP470 FB concentration in the supernatant was analyzed using the short HPLC method developed with the Agilent rapid resolution system (Example 1, Table 2).

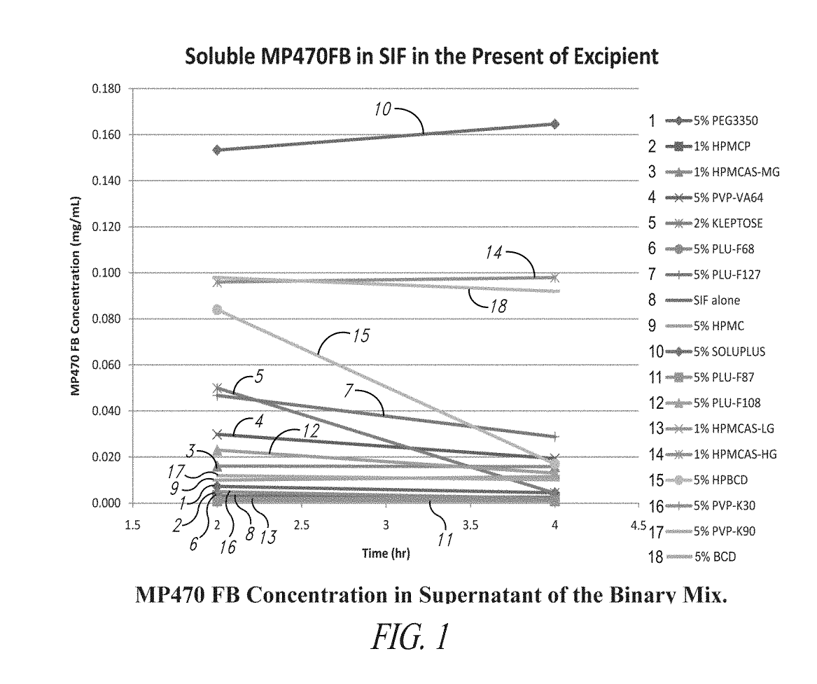

Among the polymers tested, Soluplus dissolved the most, followed by HPMCAS-HG (FIG. 1).

TABLE 5
Various Kinds of Polymers

| Excipient Category | List of Excipients |
|---|---|
| Hypromellose and | HPMC, HPMCAS (LG, MG, HG), HPMC P |
| derivatives | |
| Poloxamers | Pluronic (F68, F87, F108, F127), Soluplus |
| PEG | PEG3350 |
| Povidone and derivatives | PVP-VA64, PVP-K60, PVP-K30 |
| Cyclodextrins | β-cyclodextrin, HPBCD, MCD |

### Example 4

### Analysis of Active Ingredient Release from Soluplus in Simulated Intestinal Fluid

Soluplus was further tested for the capability to inhibit MP470 precipitation in simulated intestinal fluid (SIF). In a typical procedure, MP470 FB solution in NMP (200 mg/mL) was diluted (1/1000) into each of the Soluplus solutions pre-warmed at 37° C. to generate the solutions outlined in Table 6. The mix was incubated at 37° C. for a period of time (0.5, 2, 4, 6, 24 hr) prior to being aliquoted out for analysis of MP470 FB concentration in the supernatant. The MP470 FB concentration in the supernatant was analyzed using the short HPLC method described in Example 1, Table 2.

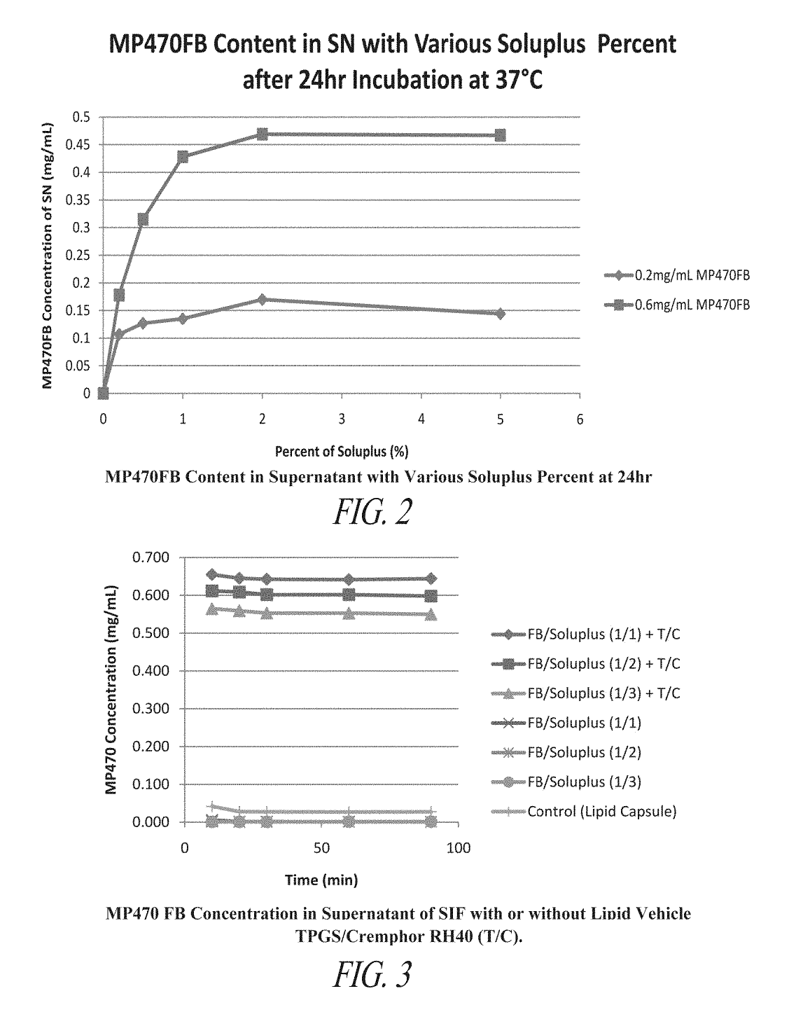

The detailed study on Soluplus revealed that interaction between MP470 and Soluplus is concentration dependent, and such interaction keeps MP470 stable in solution for at least 24 hours (Table 6 and FIG. 2).

TABLE 6
MP470FB Content in the Supernatant of Soluplus Mixture at Various
Time-points

| | 30 min | 2 hr | 4 hr | 6 hr | 24 hr |
|---|---|---|---|---|---|
| Control (SIF), MP470FB 0.2 mg/mL | 0.002 | 0 | 0 | 0 | 0 |
| 0.2% Soluplus/SIF, MP470FB 0.2 mg/mL | 0.082 | 0.084 | 0.089 | 0.087 | 0.107 |
| 0.5% Soluplus/SIF, MP470FB 0.2 mg/mL | 0.092 | 0.097 | 0.103 | 0.107 | 0.127 |
| 1.0% Soluplus/SIF, MP470FB 0.2 mg/mL | 0.096 | 0.104 | 0.115 | 0.118 | 0.135 |
| 2.0% Soluplus/SIF, MP470FB 0.2 mg/mL | 0.101 | 0.110 | 0.126 | 0.138 | 0.170 |
| 5.0% Soluplus/SIF, MP470FB 0.2 mg/mL | 0.114 | 0.124 | 0.129 | 0.137 | 0.144 |
| Control (SIF), MP470FB 0.6 mg/mL | 0.002 | 0 | 0 | 0 | 0 |
| 0.2% Soluplus/SIF, MP470FB 0.6 mg/mL | 0.126 | 0.136 | 0.152 | 0.154 | 0.178 |
| 0.5% Soluplus/SIF, MP470FB 0.6 mg/mL | 0.155 | 0.190 | 0.228 | 0.235 | 0.315 |
| 1.0% Soluplus/SIF, MP470FB 0.6 mg/mL | 0.179 | 0.223 | 0.277 | 0.318 | 0.428 |
| 2.0% Soluplus/SIF, MP470FB 0.6 mg/mL | 0.206 | 0.290 | 0.368 | 0.409 | 0.469 |
| 5.0% Soluplus/SIF, MP470FB 0.6 mg/mL | 0.244 | 0.342 | 0.374 | 0.411 | 0.467 |

### Example 5

### Analysis of Active Ingredient Release from Pharmaceutical Composition in Simulated Intestinal Fluid

Embodiments of a pharmaceutical composition were tested for the capability to inhibit MP470 precipitation in SIF. In a typical procedure, MP470 free base dispersion in Soluplus was first prepared by lyophilization at different ratios (1:1, 1:2, and 1:3). The MP470FB/Soluplus lyophile (“FB/Soluplus”) with or without 1:4 Vitamin E TPGS/Cremophor RH40 (“TIC”) was mixed with SIF solution pre-warmed at 37° C. The Control (Lipid Capsule) is MP470FB in only T/C. The mix (1 mg/mL MP470FB (total)) was incubated at 37° C. for a period of time (10 min, 20 min, 30 min, 60 min and 90 min) prior to aliquoting out a sample for analysis of MP470FB concentration in the supernatant. The MP470 FB concentration in the supernatant was analyzed using the short HPLC method described in Example 1, Table 2.

Surprisingly, when the lipid vehicle (T/C) was added to FB/Soluplus, the solid dispersion demonstrated dramatic increase (˜10 fold) of MP470 solubility over FB/Soluplus alone and T/C alone (FIG. 3). While not wishing to be bound by theory, the present applicants believe this is a result of heretofor unkown synergy between soluplus and the lipid vehicle.

In another typical procedure, MP470.HCl in T/C (“Control”) was mixed with or without Soluplus to generate a final composition of 4.4% MP470FB, 16.6% TPGS, and 66.6% Cremophor RH40 with our without 12.3% Soluplus. A sample of the formulation was mixed with SIF solution pre-warmed at 37° C. to make a 1 mg/mL MP470 (total) mix. The mix was incubated at 37° C. for a period of time (10 min, 20 min, 30 min, 60 min and 90 min) prior to aliquoting out a sample for analysis of MP470 concentration in the supernatant. The MP470 concentration in the supernatant was analyzed using the short HPLC method described in Example 1, Table 2.

When Soluplus was added to MP470.HCl in T/C (i.e., the control) the MP470 solubility was enhanced greatly (˜10 fold) (FIG. 4).

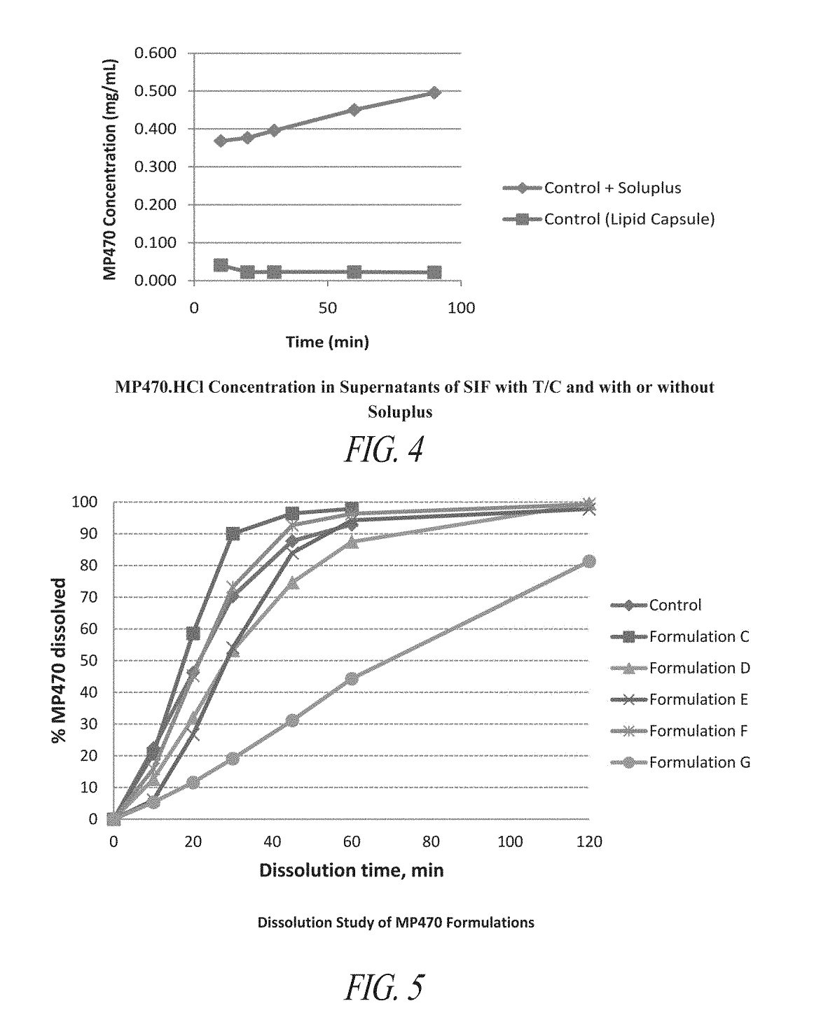

In yet another exemplary procedure, five formulations containing Soluplus and lipid vehicle (T/C) were prepared, and tested in vitro (FIG. 5). In a typical procedure, the formulations where mixed with SIF pre-warmed at 37° C. The mix (1 mg/mL MP470 (total)) was incubated at 37° C. for a period of time (10 min, 20 min, 30 min, 45 min, 60 min, and 120 min) prior to aliquoting out a sample for analysis of MP470 concentration in the supernatant. The MP470 concentration in the supernatant was analyzed using the short HPLC method described in Example 1, Table 2.

Formulation C—Crystalline MP470HCl in Soluplus in T/C, 31 mg/cap;

Formulation D—Spray dried MP470FB/Soluplus (1/1) in T/C, 31 mg/cap;

Formulation E—Spray dried MP470HCl/Soluplus (1/1) in T/C, 30 mg/cap;

Formulation F—Spray dried MP470HCl/Soluplus (2/1) in T/C, 30 mg/cap;

Formulation G—Spray dried MP470HCl/Soluplus (2/1) in T/C, 66 mg/cap;

Control—MP470.HCl clinical lipid capsules (MP470.HCl in T/C).

These examples provide evidence that the combination of Soluplus/Vitamin E TPGS/Cremophor RH40 can synergistically increase solubility of both crystalline and amorphous MP470.

### Example 6

### Analysis of Active Ingredient Release from Pharmaceutical Composition Compared to HPMCAS-HG/L/C in Simulated Intestinal Fluid

Eleven lab scale batches of spray dried MP470 powders were prepared by Formurex, using two polymers (Soluplus and HPMCAS-HG), two API (MP470 free base and MP470.HCl), and two drug/polymer ratios (Table 7). In a typical procedure, the spray dried MP470 powders were pre-mixed with lipid vehicle (T/C) and SIF solution pre-warmed at 37° C. The control contained MP470.HCl and T/C only. The mix (1 mg/mL MP470 (total)) was incubated at 37° C. for a period of time (10 min, 20 min, 30 min, 60 min and 90 min) prior to aliquoting out a sample for analysis of MP470 concentration in the supernatant. The MP470 concentration in the supernatant was analyzed using the short HPLC method described in Example 1, Table 2.

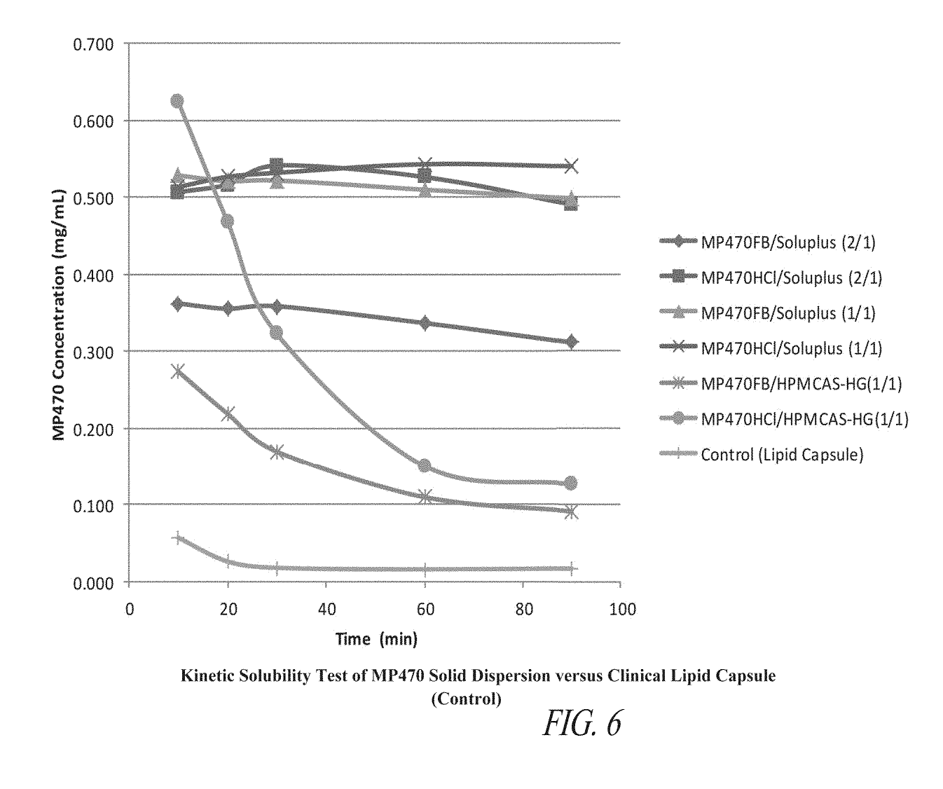

Soluplus with T/C showed higher MP470 powder solubility than HPMCAS-HG with T/C (FIG. 6).

TABLE 7
Spray Dried MP470 Powders Prepared by Formurex

| Batch # | 1 | 2 | 3 | 4 | 5 | 6 | 7 | 8 | 9 | 10 | 11 |
|---|---|---|---|---|---|---|---|---|---|---|---|
| MP470 FB | + | | + | | + | | + | | + | | |
| MP470.HCl | | + | | + | | + | | + | | + | + |
| Soluplus | + | + | | | + | + | + | + | | | + |
| HPMCAS-HG | | | + | + | | | | | + | + | |
| MP470/Polymer | 1 | 1 | 1 | 1 | 1 | 1 | 2 | 2 | 1/3 | 1/3 | 2 |
| Ratio | | | | | | | | | | | |
| Total Amount | 90 mg | <0.5 g | <0.5 g | <0.5 g | 6.9 g + | 3.6 g + | 139 mg | 214 mg | 0.9 g | 1.4 g | 1.7 g + |
| | | | | | 3.3 g | 2.1 g | | | | | 6.8 g |

### Example 7

### Analysis of Active Ingredient Release from Pharmaceutical Composition in Mammalian Model

Five formulations containing Soluplus and lipid vehicle (T/C) were prepared, and tested in vivo (Table 8). In a typical procedure, the formulations were administered in a single dose orally to 4 male dogs and blood samples were taken pre-dose and after dosing at 30 min., 1 hr, 2 hr, 4 hr, 8 hr, 24 hr, and 48 hr. All blood samples were collected into tubes containing potassium ethylenediaminetetraacetic acid and stored below—20° C. Plasma samples were extracted using a protein precipitation procedure, and MP470 was analyzed using validated liquid chromatographic-tandem mass spectrometric methods.

Formulation C—Crystalline MP470HCl in Soluplus/T/C, 31 mg/cap;

Formulation D—Spray dried MP470FB/Soluplus (1/1) in T/C, 31 mg/cap;

Formulation E—Spray dried MP470HCl/Soluplus (1/1) in T/C, 30 mg/cap;

Formulation F—Spray dried MP470HCl/Soluplus (2/1) in T/C, 30 mg/cap;

Formulation G—Spray dried MP470HCl/Soluplus (2/1) in T/C, 66 mg/cap;

Control—MP470.HCl clinical lipid capsules (T/C).

TABLE 8
PK Summary of MP470 Formulations in Dogs

| | | | Dose | | Half-Life | | Tₘₐₓ | | Cₘₐₓ | | AUCₐₗₗ | |
|---|---|---|---|---|---|---|---|---|---|---|---|---|
| | | | Mean | SD | Mean | SD | Mean | SD | Mean | SD | Mean | SD |
| Group_No | Formulation | Animal_ID | (mg/kg) | | (hr) | | (hr) | | (ng/mL) | | (hr*ng/ml) | |
| 1 | Control | M1-M4 | 10.1 | 1.2 | 6.6 | 2.4 | 1.0 | 0.4 | 404 | 186 | 2172 | 1067 |
| 2 | HCl_C | M5-M8 | 11.3 | 1.0 | 5.5 | 1.4 | 1.3 | 0.6 | 1165 | 422 | 6941 | 2528 |
| 3 | FB_SD1_D | M9-M12 | 10.3 | 0.3 | 5.0 | 2.0 | 1.1 | 0.5 | 475 | 164 | 2714 | 861 |
| 4 | HCl_SD2_E | M13-M16 | 10.7 | 1.5 | 6.9 | 2.2 | 1.5 | 1.7 | 995 | 110 | 6520 | 2691 |
| 5 | HCl_SD6_F | M17-M20 | 10.3 | 1.2 | 4.9 | 1.3 | 1.4 | 0.6 | 977 | 614 | 6549 | 4731 |
| 6 | HCl_SD6_G | M21-M24 | 7.6 | 0.9 | 4.4 | 1.3 | 1.0 | 0.6 | 673 | 217 | 3361 | 401 |

The effects of MP470/Soluplus ratio, drug loading, acidification, and two-pack kit approach on oral exposure of MP470 were also evaluated in a dog pharmacokinetics (“PK”) cross over study using nine different formulations (Table 9). In a typical procedure, dogs were given each formulation in one oral dose on separate occasions with an at least 6 day washout period. Blood samples were taken pre-dose and after dosing at 30 min., 1 hr, 2 hr, 4 hr, 8 hr, 24 hr, and 48 hr to determine serum MP470 concentrations. All blood samples were collected into tubes containing potassium ethylenediaminetetraacetic acid and stored below −20° C. Plasma samples were extracted using a protein precipitation procedure, and MP470 was analyzed using validated liquid chromatographic-tandem mass spectrometric methods.

Formulation C2A with MP470/Soluplus ratio of 2/1 had the highest bioavailability (“BA”) enhancement over the control (clinical lipid capsules) (Table 10). It appears that 50-75 mg/g (Formulations C2A and C2A_75) at MP470/Soluplus ratio of 2/1 had the highest MP470 loading strength. Acidification of the formulation (C2A_B), targeting to reduce potential free base formation during manufacturing and storage of the formulation, resulted in a drop in oral exposure compared to the unacidified formulation. The two-vial kit formulation (Vehicle+API), aimed at maintaining stability of the drug product specifically suppressing free base formation, did offer the same BA enhancement as the capsule formulation (C1A).

TABLE 9
Summary of Nine MP470 Formulations Tested in Dog Cross-over PK Study

| | Study | | | Drug:Polymer | Target Drug | |
|---|---|---|---|---|---|---|
| Phase | Day | Formulation Name | Composition (w/w) | ratio | Loading (%) | Dose/animal |
| 1 | 1 | MP470HCl_C-1A | 5.3% MP470HCl, 5.0% Soluplus, | 1:1 | 5.0 | 3 capsules |
| | | | 17.9% TPGS, 71.6% Cremophor RH40 | | | (29 mg/cap) |
| 2 | 7 | MP470HCl_C-4A | 5.3% MP470HCl, 1.3% Soluplus, | 4:1 | 5.0 | 3 capsules |
| | | | 18.7% TPGS, 74.7% Cremophor RH40 | | | (29 mg/cap) |
| 3 | 14 | MP470HCl_C-2A | 5.3% MP470HCl, 2.5% Soluplus, | 2:1 | 5.0 | 3 capsules |
| | | | 18.4% TPGS, 73.8% Cremophor RH40 | | | (30 mg/cap) |
| 4 | 21 | MP470 HCl (T/C) | 5.4% MP470HCl, 18.9% TPGS, 75.7% | n/a | 5.0 | 3 capsules |
| | | | Cremophor RH40 | | | (29 mg/cap) |
| 5 | 28 | MP470HCl_C_67 | 7.5% MP470HCl, 4.9% Soluplus, | 1.4:1 | 6.7 | 2 capsules |
| | | | 17.5% TPGS, 70.1% Cremophor RH40 | | | (41 mg/cap) |
| 6 | 35 | MP470HCl_C_75 | 8.1% MP470HCl, 7.3% Soluplus, | 1:1 | 7.5 | 2 capsules |
| | | | 16.9% TPGS, 67.7% Cremophor RH40 | | | (44 mg/cap) |
| 7 | 56 | MP470HCl_C2A_B | 5.4% MP470HCl, 2.5% Soluplus, 1.0% | 2:1 | 5.0 | 3 capsules |
| | | | Acetic acid, 18.2% TPGS, 72.9% | | | (32 mg/cap) |
| | | | Cremophor RH40 | | | |
| 8 | 65 | MP470 Aqueous | Vial A: 81.5% WFI, 1.0% Soluplus, | 1:1 | 5.0 | 90 mg |
| | | Suspension (Two-pack | 3.5% TPGS, 14.0% Cremophor RH40 | | | (10 mg/mL) |
| | | Kit Formulation) | Vial B: MP470HCl | | | |
| 9 | 77 | MP470HCl_C2A_75 | 8.2% MP470HCl, 3.8% Soluplus, | 2:1 | 7.5 | 2 capsules |
| | | | 17.6% TPGS, 70.4% Cremophor RH40 | | | (49 mg/cap) |

TABLE 10
The PK Summary of Nine MP470 Formulations in Dog Model

| | | AUCₐₗₗ, | Relative Total |
|---|---|---|---|
| | Cₘₐₓ, ng/mL | hr * ng/mL | Exposure |
| Formulation | (% CV) | (% CV) | (Enhanced/Clinical) |
| Clinical lipid capsule; 50 mg/g (No Soluplus, control) | 265 (16) | 1686 (32) | 1.0 |
| Drug:Soluplus @ 1:1, 50 mg/g (MP470HCl_C-1A) | 893 (30) | 6148 (44) | 3.6 |
| Drug:Soluplus @ 2:1, 50 mg/g, acidified (MP470HCl_C2A_B) | 683 (44) | 4073 (41) | 2.4 |
| Drug:Soluplus @ 1:1, 50 mg/g, two-pack kit formulation | 967 (36) | 6654 (18) | 3.9 |
| Drug:Soluplus @ 1.4:1, 67 mg/g (MP470HCl_C_67) | 678 (36) | 4741 (41) | 2.8 |
| Drug:Soluplus @ 1:1, 75 mg/g (MP470HCl_C_75) | 611 (39) | 4649 (51) | 2.8 |
| Drug:Soluplus @ 4:1, 50 mg/g (MP470HCl_C-4A) | 658 (35) | 4384 (41) | 2.6 |
| Drug:Soluplus@ 2:1, 50 mg/g (MP470HCl_C-2A) | 1239 (13) | 9293 (30) | 5.5 |
| Drug:Soluplus@ 2:1, 75 mg/mL (MP470HCl_C2A_75) | 1368 (59) | 8882 (53) | 5.3 |

All of the U.S. patents, U.S. patent application publications, U.S. patent applications, foreign patents, foreign patent applications and non-patent publications referred to in this specification are incorporated herein by reference, in their entirety to the extent not inconsistent with the present description.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A pharmaceutical composition comprising:
a) amuvatinib, or a stereoisomer, tautomer, pharmaceutically acceptable salt or prodrug thereof;
b) a surfactant polymer, or a pharmaceutically acceptable salt thereof;
c) a tocopherol, or a pharmaceutically acceptable salt thereof; and
d) a fatty acid, a fatty acid ester, or a pharmaceutically acceptable salt thereof.

2. The pharmaceutical composition of claim 1, wherein the surfactant polymer comprises a polyoxyalkylene.

3. The pharmaceutical composition of claim 2, wherein the surfactant polymer comprises a polyoxyalkylene, a polyvinyl lactam and a polyvinyl ester.

4. The pharmaceutical composition of claim 3, wherein the surfactant polymer has the following structure (II): 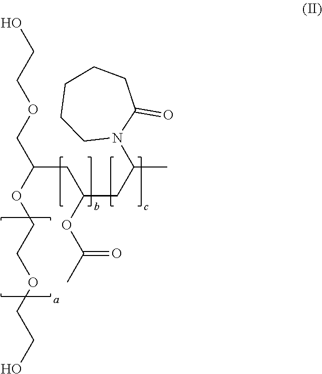
or a pharmaceutically acceptable salt thereof, wherein a, b and c are integers greater than one and a, b and c are selected such that the surfactant polymer has an average molecular weight, as determined by gel permeation chromatography, ranging from about 90,000 g/mol to about 140,000 g/mol.

5. The pharmaceutical composition of claim 1, wherein the tocopherol is α,β, γ or δ tocopherol, or a derivative or ester thereof.

6. The pharmaceutical composition of claim 1, wherein the tocopherol is d-alpha tocopherol polyethylene glycol 1000 succinate.

7. The pharmaceutical composition of claim 1, wherein the pharmaceutical composition comprises a polyalkylene oxide fatty acid ester, or pharmaceutically acceptable salt thereof.

8. The pharmaceutical composition of claim 7, wherein the polyalkylene oxide fatty acid ester is an ester of a long chain fatty acid.

9. The pharmaceutical composition of claim 8, wherein the long chain fatty acid is stearic acid or ricinoleic acid or hydrogenated or hydroxylated derivatives thereof.

10. The pharmaceutical composition of claim 1, wherein the fatty acid or fatty acid ester is glycerol polyethylene glycol 12-hydroxystearate (Cremophor RH40), polyoxyl 15 12-hydroxystearate (Solutol HS 15) or PEG 20 stearate (Lipopeg 10-S).

11. The pharmaceutical composition of claim 1, wherein a ratio of amuvatinib to the fatty acid, the fatty acid ester, or a pharmaceutically acceptable salt thereof is about 1:15

12. The pharmaceutical composition of claim 1, wherein a ratio of amuvatinib or pharmaceutically acceptable salt thereof to the surfactant polymer ranges from about 1:0.1 to about 0.1:1.

13. The pharmaceutical composition of claim 1, wherein the amuvatinib or pharmaceutically acceptable salt thereof is present in the pharmaceutical composition in a mass percentage ranging from about 1% to about 10%.

14. The pharmaceutical composition of claim 1, wherein the pharmaceutical composition further comprises one or more other chemotherapeutic agents.

15. The pharmaceutical composition of claim 14, wherein the chemotherapeutic agent is selected from mitotic inhibitors, alkylating agents, anti-metabolites, cell cycle inhibitors, enzymes, topoisomerase inhibitors, biological response modifiers, anti-hormones, antiangiogenic agents, anti-androgens, platinum coordination complexes, substituted ureas, methylhydrazine derivatives, adrenocortical suppressants, hormone and hormone antagonists, progestins, estrogens, antiestrogens, androgens, and aromatase inhibitors.

16.-18. (canceled)

19. A method for the treatment of cancer, the method comprising administering to a subject in need thereof a therapeutically effective amount of the pharmaceutical composition of claim 1.

20. A method for the treatment of a protein kinase-mediated disease, the method comprising administering to a subject in need thereof a therapeutically effective amount of the pharmaceutical composition of claim 1.
